(19) United States
(12) Patent Application Publication
(10) Pub. No.: US 2018/0093083 A1
(43) Pub. Date: Apr. 5, 2018

(54) APPLICATOR TOOL AND METHODS OF USING THE SAME

(71) Applicant: Jessica R. Headington, Ashburn, VA (US)

(72) Inventors: Jessica Ros Headington, Ashburn, VA (US); Scott D. Balderston, Jeffersonton, VA (US)

(73) Assignee: Jessica R. Headington, Ashburn, VA (US)

(21) Appl. No.: 15/286,244

(22) Filed: Oct. 5, 2016

(51) Int. Cl. A61F 13/40; A41D 19/00; A45D 33/34

(52) U.S. Cl. CPC A61M 35/006; A45D 33/34; A41D 19/0024

(57) ABSTRACT

Implementations relate to an applicator tool and methods of using the same. Implementations relate to an applicator tool and related techniques used to apply cosmetic material, medicines, bandages, or other medical material, or other materials using a glove configured with a set of applicator pads. In cases, the set of applicator pads can be affixed to the individual fingers of the glove or other support. A user can put the applicator tool on one hand or use one on each hand. The set of applicator pads can be formed of sponge or other material which can acquire cosmetic foundation or other material with a dabbing action in a container for the desired material. The cosmetic or other material can then be applied with a bouncing, dabbing or tapping action on the cheek or other area of the skin or body. Dragging and smearing across the skin can be avoided.

### FIELD

The present teachings relate to an applicator tool and methods of using the tool, and in particular implementations, to an applicator tool for applying cosmetic products and other materials to the skin of a user.

### BACKGROUND

In the field of cosmetics and beauty products, users have become accustomed to applying various powders, gels, creams, liquids, emulsions, and other products or materials to desired areas of the face or skin using commonly available tools. Online video sources, such as YouTube™ or others, contain many instructional or tutorial videos demonstrating the selection and use of cosmetic products, the tools used to apply those products, and techniques for applying those products. Aided by those instructions, as well as manufacturer media and advertising, more consumers than ever have learned how to apply cosmetic products of their choice. A great many cosmetic products and techniques are available, and are heavily marketed to young female consumers and other segments of the marketplace.

A common and basic cosmetic product is foundation. Foundation is generally a type of preparation used to provide a smooth base texture for the application of additional layers of coloring agents or other cosmetic material. For many users of cosmetic products, a frequent if not daily routine begins with the application of a base foundation layer, followed by other layers of selected cosmetic material. Following an effective foundation routine is essential to achieving a number of desired cosmetic effects with good results.

Cosmetic foundation can be compared to wall paint primer, which likewise serves as a base layer and similarly comes in a wide range of colors and tones to establish a foundation for additional layers. Foundation generally consists of a skin-colored makeup that comes in a liquid, press powder, loose powder, cream, or liquid form. Foundation typically varies in coverage on the skin, from light to full coverage, depending on one's preferences. Foundation is generally applied to the face to help cover up blemishes, even out surface complexion, blur spots, or to modify one's skin tone. Foundation is used all over the face to even out the complexion. One goal of foundation is to make one's skin appear to have a smooth, nearly flawless, or airbrushed appearance.

To achieve a nearly flawless appearance with poreless, radiant, and natural-looking skin, it is important to expertly and carefully blend the foundation makeup into the skin. A traditional way to do this is to buff the foundation into the skin. This is frequently done using a makeup applicator brush. An applicator brush usually consists of a cluster of brush strands, often made of synthetic material, mounted in a small handheld base. The user uses the brush to rub or brush over foundation material, which can be provided in a small shallow cosmetic container or dispenser. Small amounts of the foundation material adhere to the brush strands. After the brush is loaded with foundation material, the user takes the brush and applies the brush strands to their cheek or other facial area they wish to prepare and cover with foundation. The user gradually works the foundation material in place with side to side or circular motions, to serve as the base for other layers of cosmetic treatments.

Brush-based application of foundation and other cosmetic materials has, however, a number of drawbacks for many users. For one, the brush strands, even when manufactured in a fairly fine or narrow width, may often acquire too much material which is not effectively transferred to the skin, resulting in waste. In addition, the most active contact area for the skin is the tips of the bristles or strands, and cosmetic material can be acquired but remained trapped or stuck along the length of the bristles. This can produced additional waste of material since that part of the bristles may not contact, or may not fully contact, desired areas of the face or skin.

As another disadvantage, when using a brush tool, the user as noted generally performs an action where the brush strands are dragged, swiped, or stroked across the skin in a sideways or circular motion. With that kind of tugging or pulling action, the brush strands, when brushed across the skin, can tend to be slightly abrasive and tug, pull, or stretch the user's skin. This can irritate the skin, and cause potential complications such as acne, reddening or inflammation of the skin, or others. For example, frequent use of an applicator brush can cause unintended sagging and premature aging of the skin tissue.

The use of an applicator brush to buff foundation into place is also a relatively slow process, which can allow the foundation or other material to dry before the user is able to evenly distribute the product on desired areas of the face or skin. As the product dries, one must tug or bush even harder on the skin, to try to evenly distribute the product.

In addition, the abrasiveness of the brushing action can cause an uneven distribution of the foundation or other material. When foundation is not smoothly applied, the user may have to go back and apply more foundation in deficient areas to even out the foundation layer. This can be wasteful, and result in a foundation layer that is thicker or thinner than desired in different locations of the face or skin.

Still further, the effective use of applicator tools, and achieving the desired makeup effects, depends at least partly on the user having good tactile sensation as they apply the cosmetic material. Good fingertip sensation helps the user feel bare or shallow spots in the foundation as material is built up, as well as contours of the skin, cheek bones, eyebrows, or other surfaces or structures as the application progresses.

A user for example may not be able to accurately contour the face using an applicator brush. Contouring is the process of adding darker powder (e.g. bronzer) to the hollows of the cheekbones, and dusting a lighter shade under the eyes. This play with colors and shadows can sculpt the face to achieve desired effects, such as making the face look slimmer or enlarging the eyes. To apply a contouring bronzer, the user must first feel their face for the natural hollows of the cheeks, and then use the brush to apply the bronzing powder.

This technique is not always efficient using an applicator brush, because it is difficult or impossible to feel the hollows of the cheeks through a makeup brush. This leads to makeup mistakes such as applying bronzer too low on the cheeks, which elongates the face and creates a droopy effect. A brush-based tool simply does not afford the user much, if any, tactile feedback as material is applied.

Moreover, a brush-based tool may accumulate dust, dirt, dead skin cells, and other debris in the bristles which provide an unintended habitat for bacteria or other germs to accumulate and grow. Contamination of this type can likewise contribute to acne, breakouts, or other skin conditions. The applicator brush itself at the same time can be hard to clean, disinfect, or sterilize, including by using washing machines.

Yet further, because cleaning or sterilization of an applicator brush may not be easy to perform, it may not be easy or convenient to remove one foundation or other material from an applicator brush, and replace it with a new material when the user wishes to switch to a different product to apply.

Using a standard makeup brush to apply foundation, contour, or other cosmetic materials can therefore overall be unhygienic, inefficient, inaccurate, harmful or unhealthy, and can cause aging, acne, or other undesirable effects.

In addition to conventional brush-based applicator tools, the use of free-standing applicator sponges has also been known in cosmetic applications. Such individual sponges allow the user to pick up and manipulate a pre-formed sponge to dab and acquire cosmetic material, and then use the sponge to apply the material to desired areas of the face and/or skin. The use of a free sponge tool, however, requires the user to grasp or pinch that sponge between two or more fingers to manipulate and apply the desired material, which limits the user to one sponge and one material at a time.

The sponge moreover can easily slip while being held between the user's fingers, resulting in smeared or inaccurate application of foundation or other material. Furthermore, a number of available cosmetic sponges are water repellant, or absorb all of the makeup supply. Another downside to individual sponges is that they must be hand washed, rather than machine washed, which can take more time and be less effective.

It may be desirable to provide applicator tool and methods of using the same, in which a sponge-based or other applicator pad or surface is mounted or affixed to a glove or other support to allow direct manual application of cosmetic or other material with good tactile properties and other benefits, including the ability to be readily cleaned, sterilized, and reused.

### DESCRIPTION OF DRAWINGS

The accompanying drawings, which are incorporated in and constitute a part of this specification, illustrate implementations of the present teachings and together with the description, serve to explain features and principles of the present teachings. In the figures:

FIG. 1 illustrates an example of an overall configuration of an applicator tool which can be used in applicator tool and methods of using the same, according to various implementations of the present teachings;

FIG. 2 illustrates an example of an overall configuration of an applicator tool in another perspective view, according to various implementations;

FIG. 3 illustrates an example of an overall configuration of an applicator tool in another perspective view, according to various implementations;

FIG. 4 illustrates an example of an overall configuration of an applicator tool in another perspective view, according to various implementations;

FIG. 5 illustrates an applicator tool including one type or configuration of attachment between finger sections of the applicator tool and a set of applicator pads, according to various implementations;

FIG. 6 illustrates an applicator tool including one type or configuration of attachment between finger sections of the applicator tool and a set of applicator pads, according to various further implementations;

FIG. 7 illustrates an applicator tool including one type or configuration of attachment between finger sections of the applicator tool and a set of applicator pads, according to various further implementations;

FIG. 8 illustrates use of an applicator tool according to various implementations and application techniques, according to various implementations of the present teachings; and

FIG. 9 illustrates use of an applicator tool according to various further implementations and application techniques, according to various implementations of the present teachings in further regards.

### DESCRIPTION OF IMPLEMENTATIONS

Implementations of the present teachings relate to applicator tool and methods of using the same. More particularly, implementations relate to apparatus, tools, constructions, and techniques for an applicator tool, in which one or more applicator pads are attached to the fingers of a glove, or other attachment points of a support or base, to allow direct, convenient, and effective application and manipulation of cosmetic or other materials.

Reference will be made in detail to exemplary implementations of the present teachings, which are illustrated in the accompanying drawings. Where possible the same reference numbers will be used throughout the drawings to refer to the same or like parts.

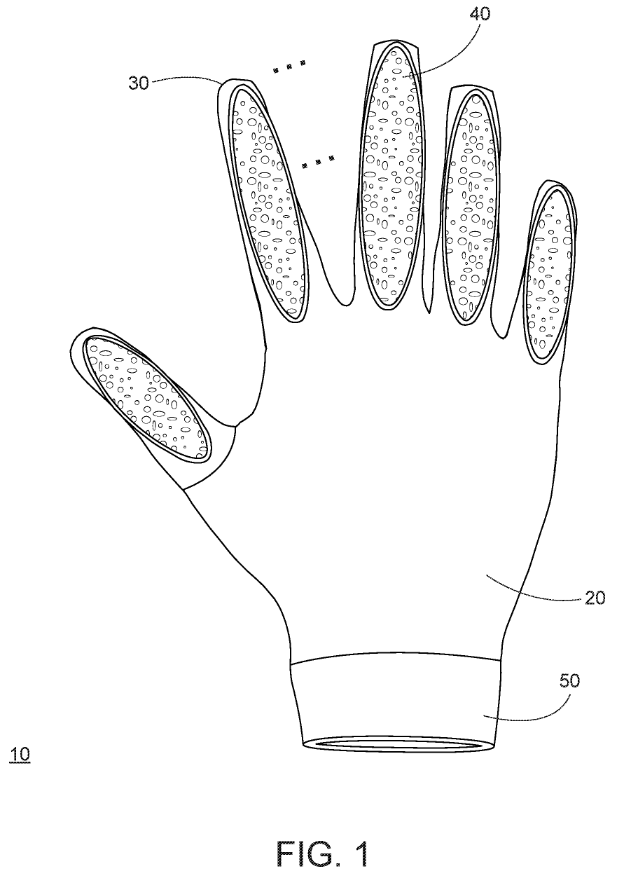

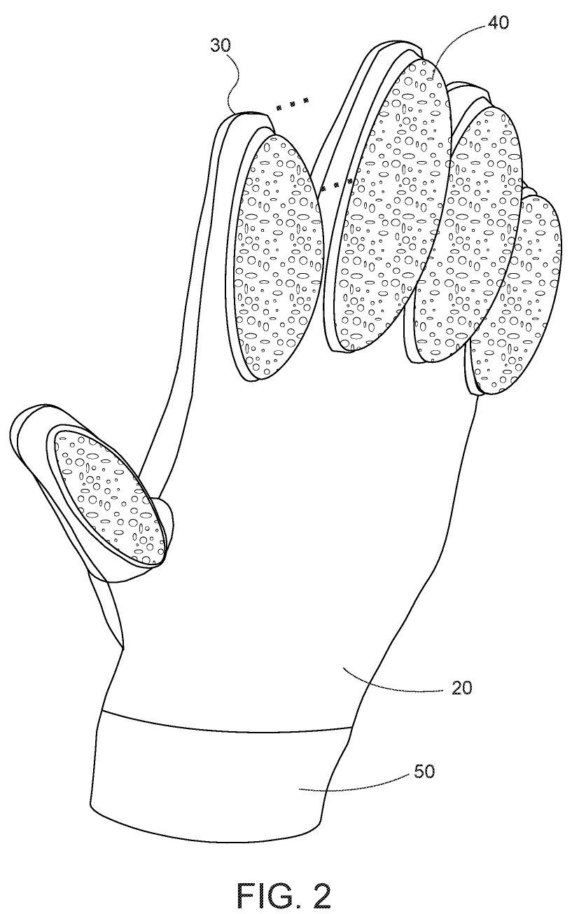

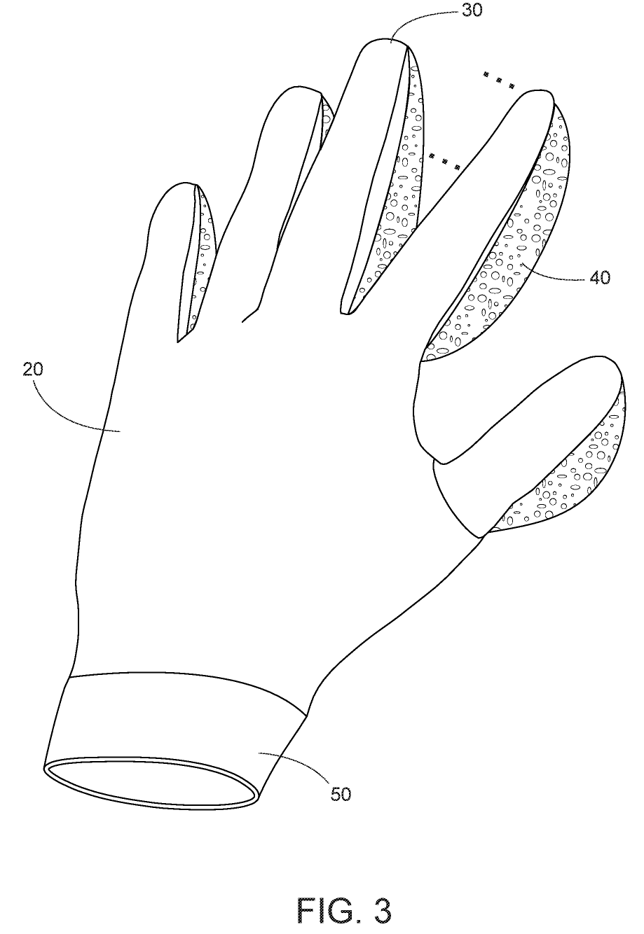

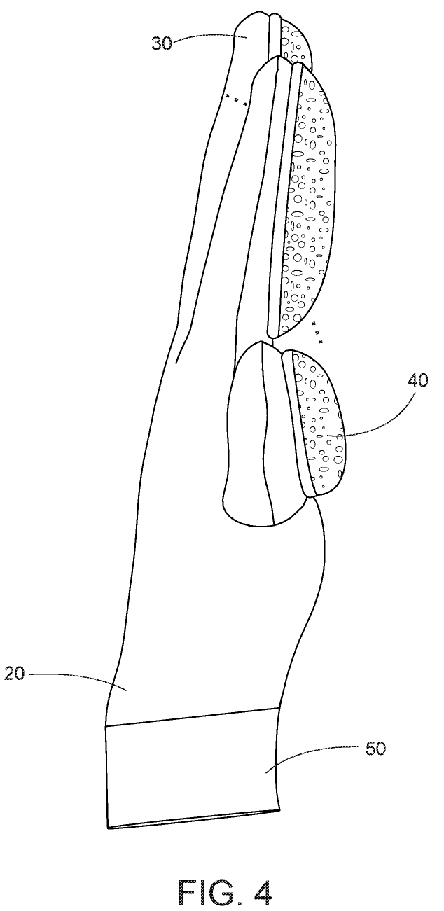

FIG. 1 illustrates an applicator tool 10, according to aspects of the present teachings. In aspects as shown in FIG. 1 and in various views of shown in FIGS. 2-4 and elsewhere, the applicator tool 10 can include a support 20. In general, FIG.1 depicts the applicator tool 10 in a frontal, or pam-forward view. FIG. 2 shows the applicator tool 10 in a partly rotated view, with the palm forward at an angle. FIG. 3 shows the applicator tool 10 from a back perspective, while FIG. 4 shows the applicator tool 10 in a side view, taken from the thumb side of a user's hand.

The support 20 shown in those and other figures can, in general, be or include any base, structure, enclosure, brace, sleeve, prop, reinforcement, garment, or mitt which a user can place on their hand and perform manipulations of desired cosmetic or other material. In implementations as shown, the support 20 can be or include a glove or glove-like enclosure fitting over a user's hand.

In implementations as shown in FIG. 1 and other figures, when formed as a glove or other wearable accessory, the support 20 can include a set of finger sections 30, to allow the user to put the glove on and insert their fingers into individual tube-like sections for their fingers. While the set of finger sections 30 are illustrated in FIG. 1 and elsewhere with five generally similar sections to receive five individual fingers, it will be appreciated that other numbers or configurations of the finger sections can be used in different implementations.

The support 20 can be made of a variety of materials, or layers or combinations of materials. For instance, the support 20 can be made of a latex material, allowing a degree of stretch and form fitting to the user's hand. Latex material when used for support 20 also provides a water-resistant barrier to protect the hand. In implementations, the support 20 can also or instead be made of a rubber material, such as a nitrile rubber or butyl rubber, or other forms or types of rubber material. In implementations, for instance, the support 20 can also or instead be made of vinyl material. In further implementations, the support 20 can be made of various types of fabric, textile, and/or other fiber material, such as cotton or wool.

In further implementations, the support 20 can be made of synthetic material, such as polyester, rayon, Spandex™, and/or other synthetic fabrics, weaves, or materials. Combinations of any of the foregoing materials can likewise be used, for example, a support 20 such as a glove made with a fabric base impregnated with rubber or other material. Layers of different materials can also or instead be used. The material or materials of the support 20 can be manufactured with antimicrobial treatments, and/or treatments against mold, mildew, or other contaminants. When made or impregnated with latex, rubber, and/or other materials, the support 20 can provide anti-slip properties as well.

In implementations, the support 20 can optionally include an elastic cuff 50 so the user can wear and use the support 20 more securely. Other arrangements, such as a fastenable wrist strap, such as an opposed hook and loop (e.g. Velcro™) strap, can also be used to secure the applicator tool 10 more securely to the user's hand or arm. While implementations are illustrated which include an elastic cuff 50, it will be appreciated that in implementations, a cuff or other attachment or tightening device can be omitted from the applicator tool 10.

In implementations, the support 20 as noted can be constructed of various internal layers, and/or provided with various treatments. For example, the interior of the support 20 can be provided with an internal liner, such as a liner made of fleece, cotton, or other material. In implementations, the interior of support 20 can also or instead be pre-treated with an application of one or more materials. For example, the interior of the support 20 can be treated with a dusting or coating of talc or other friction-reducing material, to make putting the support 20 on or taking the support 20 off easier or more convenient for the user.

In further implementations, the interior of the support 20 can be treated with a moisturizing material, such as a skin balm or emollient, to soften the user's skin during and after the use of applicator tool 10. Other treatments or materials can be used to render the putting on, taking off, and/or use of the applicator tool 10 more convenient, comfortable, safe, and/or hygienic for the user. A user can also or instead apply skin moisturizer or other skin preparations to their hands before using the applicator tool 10, to take advantage of the natural warming and moistening effects provided by an enclosed support 20.

While the support 20 is shown in FIG. 1 and other figures in the form of a glove, it will likewise be appreciated that in implementations, the support 20 could be configured in other enclosed shapes. For example, the support 20 could be constructed as a mitten without individual fingers, a support 20 having one section for enclosing two fingers and another section enclosing three fingers, or other shapes or configurations.

In terms of applicator contact surfaces, as likewise shown in FIG. 1 and other figures, the applicator tool 10 can include a set of applicator pads 40 to acquire and apply cosmetic or other materials. In implementations, the set of applicator pads 40 or tips can be or include one or more pieces of sponge or sponge-like material, formed as pads attached to the support 20. When a sponge or sponge-like material is used, the set of applicator pads 40 can for instance be constructed of latex-free or other polyurethane, to make the sponge material hypoallergenic and non-toxic. In implementations, the set of applicator pads 40 can include one pad, for instance attached to one finger section, or can include two, three, four, five, or other numbers of pads.

The sponge or other material of the set of applicator pads 40 can in cases have a foamy texture which allows the set of applicator pads 40 to be infused, pre-treated, or pre-soaked with various materials, preparations, or treatments. In implementations, for example, the set of applicator pads 40 can be infused, pre-treated, or pre-soaked with various minerals, collagen, vitamins, and/or other materials to soothe, hydrate, and/or aid in blending and application of cosmetic or other materials. In implementations, those or other soak preparations, materials, or formulates can be applied or re-applied to the set of applicator pads 40 from a separate spray bottle to mist onto the set of applicator pads 40, as the applicator tool 10 is used.

According to various uses of the applicator tool 10, the set of applicator pads 40 can be dampened or moistened before applying cosmetic or other material to prevent over-absorption of material (which is another common issue with conventional applicator brushes). The set of applicator pads 40 can be moistened or dampened simply by placing the set of applicator pads 40 under running water, by applying mist from a spray bottle, and/or other techniques. A user can squeeze out excess water in the set of applicator pads 40 if desired.

In implementations as shown, one applicator pad can be fixed, attached, or mounted to one finger section, but it will be appreciated that other configurations, numbers, or arrangements of the set of applicator pads 40 can be used. For instance, in implementations, one or more additional or separate applicator pads can be affixed or attached to a palm area of support 20, to a back of the hand area of support 20, to the knuckle area of the support 20, and/or other locations. In applications, separate or additional pads located on the palm, back of hand, and/or other locations can be used to provide additional or stored materials, such as one or more pads soaked or imbued with different types of cosmetic material, and/or soaps or other cleaning materials. Such additional pads can, in cases, be or include Velcro™ or other detachable pads, as described herein.

While the set of applicator pads 40 in FIG. 1 and elsewhere are illustrated as being of generally similarly size, length, width, thickness, and/or proportions, it will be appreciated that in implementations, any one or more of the set of applicator pads 40 can be constructed in a different manner than the remainder of the set of applicator pads 40, if desired. For example, an applicator pad located on a thumb of the set of finger sections 30 can be made smaller than the remaining pads in set of applicator pads 40, or can be omitted if desired. Similarly, the applicator pad located on a ring finger or pinky finger can be made smaller or more slender than other pads in the set of applicator pads 40, since those fingers may apply less pressure or be used less frequently than other fingers. Other different sizes, shapes, widths, thicknesses, proportions, and/or other properties can be used for any one or more pad in the set of applicator pads 40.

In addition, while in implementations shown in FIG. 1 and elsewhere the set of applicator pads 40 are illustrated as having a unitary or single-layer construction, in implementations, any one or more of the pads in the set of applicator pads 40 can be constructed in a multipart or multilayer construction. For example, one or more pads in the set of applicator pads 40 can be constructed with two, three, or other numbers of layers. For example, a pad can be constructed with a base layer made of denser or heavier sponge material to provide firmness at the attachment point to the set of finger extensions 30, with the addition of a lighter or less dense layer of sponge material as an upper layer to acquire cosmetic or other material more readily. Other configurations, including other numbers or types of layers or materials in layers, can be used in the construction of the pads in the set of applicator pads 40.

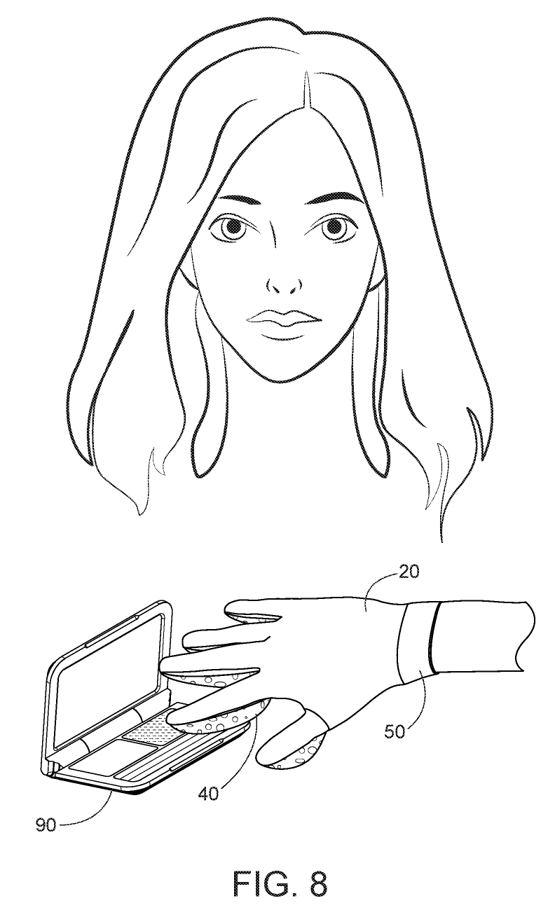

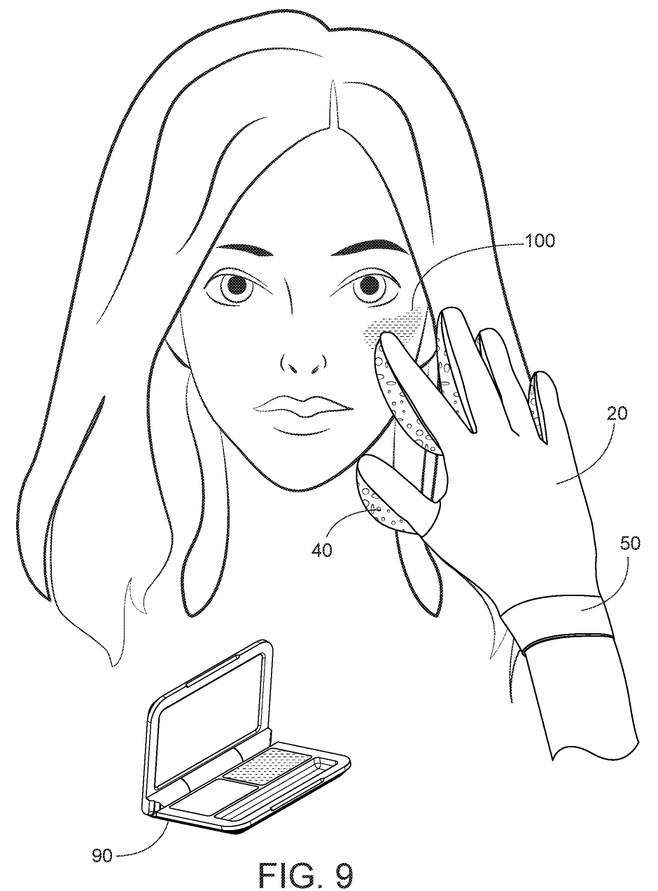

In terms of the use of the applicator tool 10 to apply cosmetic material or other material, and as for example shown in FIGS. 8 and 9, the set of applicator pads 40 can generally be touched, dabbed, dipped, or brushed across a supply container 90 of cosmetic or other material, such as a case of foundation material. One or more of the individual pads in the set of applicator pads 40 can therefore absorb, acquire, pick up, or become impregnated with the foundation material, in part due to the absorbent or adherent nature of the set of applicator pads 40. The user can, if desired, acquire material on just one, or if desired, two or more of the set of applicator pads 40. The user can, if desired, load or acquire the same foundation or other material on each applicator pad or tip, or can load or acquire different materials on different pads, to permit selective application of different materials on different fingers.

As shown for example in FIG. 9, once loaded with the desired cosmetic material, the set of applicator pads 40 can be dabbed, bounced, tapped, and/or otherwise applied to a desired area 100 of the user's skin, face, and/or other location. In general, the user may apply the material with fairly short, direct bouncing actions perpendicular or roughly perpendicular to the desired area 100. Because no dragging or tugging effect is produced by that application action, smudges, streaks, or smears in the applied material can be avoided.

In implementations, the set of applicator pads 40 can be pre-treated with selected materials such as moisteners, if desired. In implementations, the set of applicator pads 40 can also or instead be pre-treated with anti-bacterial substances or other sterilizing agents, to help enhance hygiene and safety when using the applicator tool 10. In implementations, the set of applicator pads 40 can be pre-treated with selected foundation or other cosmetic materials, if desired. In implementations, other types of materials can be pre-applied to the set of applicator pads 40, if desired.

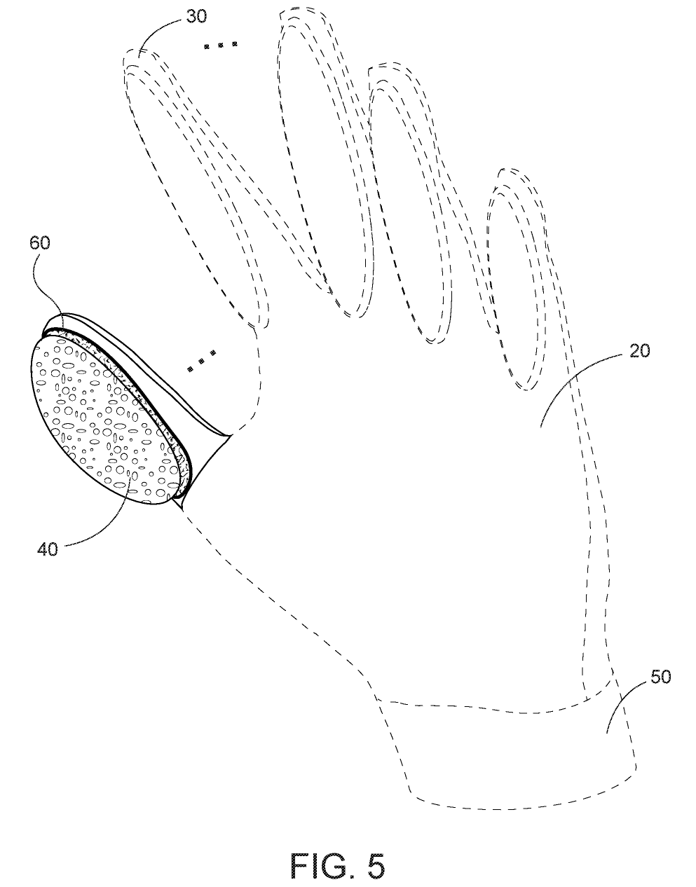

In terms of the construction of the applicator tool 10, the set of applicator pads 40 can be connected, affixed, mounted, or attached to the support 20 in a variety of ways. As shown in FIG.5, for instance, each pad of the set of applicator pads 40 can be attached to the support 20 using an adhesive 60. The adhesive 60 can be or include glue, epoxy resin, acrylic resin, silicone-based material, and/or other adhesive material.

In implementations, the adhesive can be applied to the entire backing area of each pad of the set of applicator pads 40, and be cured in place to permanently attach the set of applicator pads 40 to the set of finger sections 30 or other areas of the support 20. In implementations, the adhesive can be applied to an outer edge or peripheral area of the set of applicator pads 40, to form a line or bead to attach the set of applicator pads 30 to the set of finger sections 30 or other areas. Other patterns can be used in the application of adhesive 60, as well. In implementations, multiple layers of an adhesive 60, or two or more adhesives, can be used. In aspects, it may be useful to make the layer of adhesive 60 as thin as practicable, to help preserve the tactile sensations experienced by the user wearing the applicator tool 10.

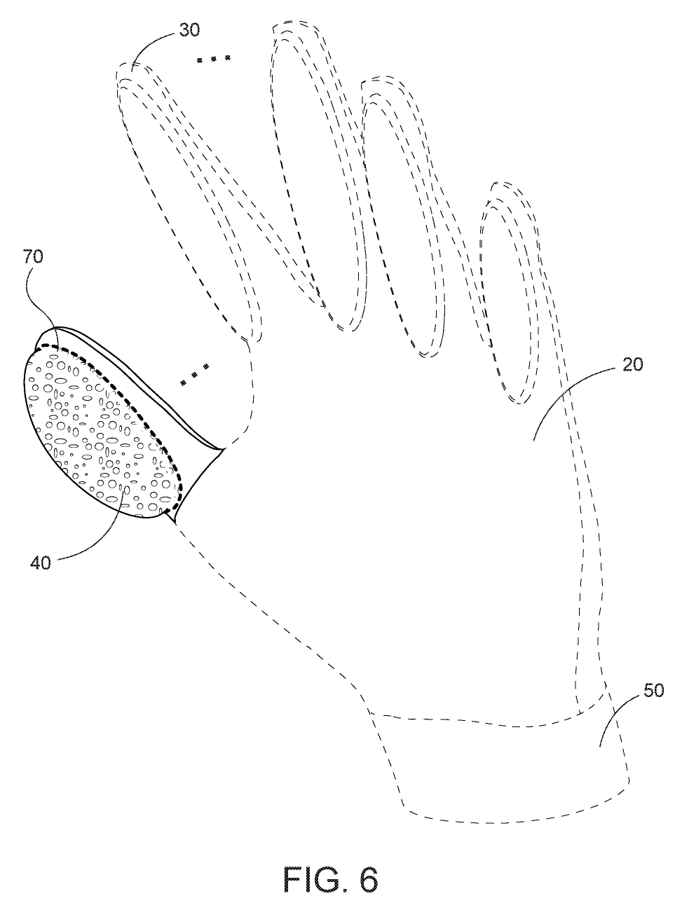

According to the present teachings in further regards, and as for instance shown in FIG. 6, the set of applicator pads 40 can also or instead be attached to the finger sections or other areas of the support 20 using stitching 70. The stitching 70 can for example be or include single-strand or bundled strands of synthetic or other thread material. In implementations as shown, the stitching 70 can be applied in over-and-under fashion to the material of the support 20 and the perimeter of the material of the set of applicator pads 40. It will be appreciated however that other patterns or layouts of stitching 70 can be used.

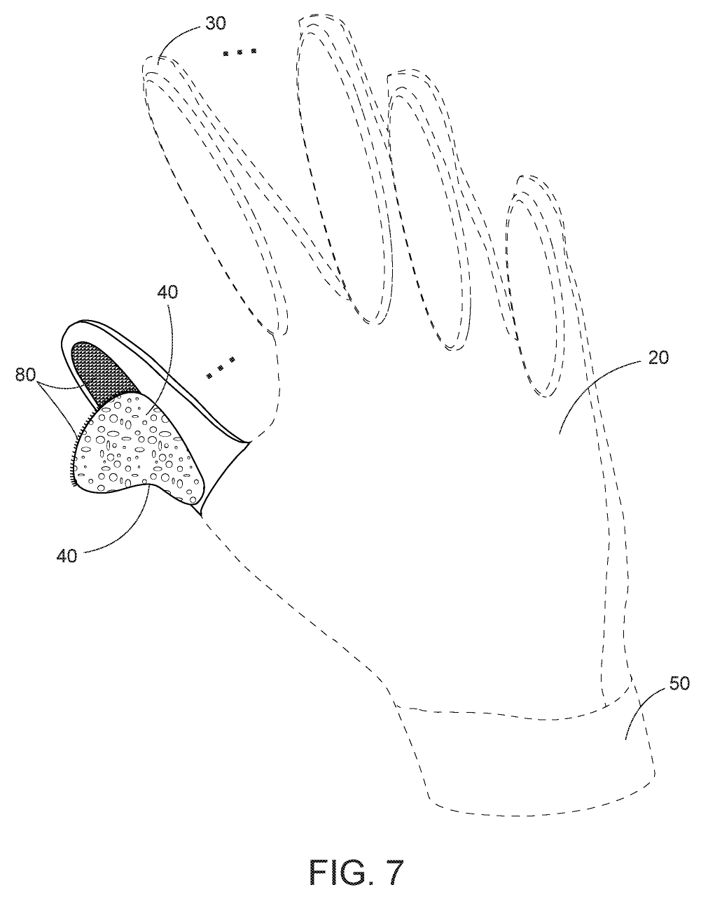

According to the present teachings in additional regards, and as for instance shown in FIG. 7, the set of applicator pads 40 can also or instead be attached to the finger sections or other areas of the support 20 using a pair of opposed hook-and-loop surfaces 80. The opposed hook-and-loop surfaces 80 can be or include, for instance, a pair of Velcro™ contact areas. In that or other type of hook-and-loop arrangement, small plastic hook members or fibers can engage with small loop members to create a cumulative binding force which is sufficient to hold the set of applicator pads 40 in firm attachment to the applicator tool 10 The opposed hook-and-loop surfaces 80 can be attached to the applicator tool 10 and set of applicator pads 40 using for instance adhesive mounting, or can in addition or instead be formed or molded into the applicator tool 10, set of applicator pads 40, or both.

When opposed hook-and-loop surfaces 80 such as Velcro™ materials or others are used, the set of applicator pads 40 as a further feature can be used in an attachable/detachable fashion. For example, the set of applicator pads 40 can be detached or removed for washing, cleaning, or replacement. In further regards, when opposed hook-and-loop surfaces 80 are used, different types, shapes, sizes, or configurations of the set of applicator pads 40 can be used for different fingers or other areas of the applicator tool 10. One set of set of applicator pads 40 can, for instance, be configured for application of foundation material, while different sized or shaped applicator pads can be configured for application of blush, mascara, and/or other products. The use of opposed hook-and-loop surfaces therefor permits the ready substitution or replacement of the set of applicator pads 40, according to user the user's desired application, selected application materials, and/or other factors. While opposed hook-and-loop surfaces 80 have been described, in implementations, other types of detachable connections between applicator tool 10 and set of applicator pads 40 and/or other parts of the applicator tool 10 can be used.

It will be appreciated that while different types of permanent or detachable mountings or connections between applicator tool 10 and set of applicator pads 40 are shown in FIGS. 5-7, other types of construction or attachment can be used. For example, in implementations, the set of applicator pads 40 can be affixed to the finger sections or other sections of the applicator tool 10 using laser welding, heat-press welding, and/or other types of attachment or construction.

In terms of general use of the applicator tool 10, again, after the user has placed the applicator tool 10 on their hand and acquired the cosmetic or other material to the set of applicator pads 40, the user can begin to apply that material to desired areas of the face or skin. Typical user actions or activity that can be used in applying the desired material are shown for instance in FIGS. 8 and 9. In aspects, the user can apply or deliver the material using repeated bouncing, dabbing, or tapping actions against the skin. A small amount of the foundation material, or other cosmetic or other material, can be applied, delivered, or deposited with each contact with the skin. The user can repeat the bouncing, dabbing, or tapping actions over desired areas of the face or skin, to produce a building-up of the cosmetic or other material.

It may be noted that because the applicator tool 10 does not require that the set of applicator pads 40 be dragged across the skin, stretching and irritation of the skin can be avoided during use. In addition, the bouncing, dabbing, or tapping action can act to stimulate blood flow through the treated areas, causing a massage-like effect for the user's face or skin. Those effects can likewise stimulate collagen production, the absorption of oxygen, blood perfusion, and enhance skin health generally. Skin wrinkles and age spots for example can be suppressed or reduced.

Furthermore, because the set of applicator pads 40 are attached to finger or other sections of the support 20, the user can be provided with greater tactile feedback as they work the cosmetic or other material into the skin. Greater evenness of the material, as well as greater precision of coloring and other effects on desired skin areas, can consequently be achieved.

If the user begins with the application of a foundation layer, the user can continue by applying a further layer of blush, coloring, or other types of cosmetic material. If desired, the user can place the applicator tool 10 under running water and wash or rinse unused materials away before acquiring material for further layers. When the user has applied all desired layers or materials, the user can place the applicator tool 10 under running water again, and/or toss the applicator tool 10 into a washing machine or similar, to rinse and clean the applicator tool 10 for additional use. The applicator tool 10 can be cleansed with mild soap or detergent, and/or other agents in a washing machine or under running water if desired.

In use, it will be noted that the user in cases can choose to use one applicator tool 10 on one hand, or can choose to use two of the applicator tool 10, one on each hand, if she or he desires.

Among other features and advantages, a user of the applicator tool 10 can therefore use foundation or other cosmetic or other products or materials with greater precision, comfort, and a greater number of potential healthy skin benefits compared to conventional brush-based applicator tools. Less waste of cosmetic or other material may also occur, and the washable nature of the applicator tool 10 allows for economical long-term use. In implementations, the applicator tool 10 can be reused many times by the same or different users. Depending on conditions of use and other factors, the applicator tool 10 can be expected to have a useful life of about 3 months, and/or lesser or greater amounts of time.

The foregoing description is illustrative, and variations in configuration and implementation may occur to persons skilled in the art. For example, as noted, while implementations have been described in which a single applicator tool 10 is worn on one hand to apply foundation or other cosmetic materials, in implementations, the user can wear an applicator tool 10 on each hand.

Similarly, and as noted above, while applicator tool 10 has been described generally in connection with the application and use of cosmetic materials, in implementations or applications, the applicator tool 10 can be used to acquire and apply other types of materials. For instance, the applicator tool 10 can also be used in medical applications. The applicator tool 10 can for example be used to apply analgesic liquids, creams, or gels to topical skin areas. Similarly, the applicator tool 10 can be used to safely and effectively apply antibiotic materials to target skin areas, for instance, to treat skin infection, skin cuts, bleeds, abrasions, or burn areas. Other medicines, preparations, or medical materials can be used. For instance, the applicator tool 10 can be used to apply a liquid, gel, or cream preparation used to form a self-curing or self-drying bandage or other topical protection for a selected skin area.

In further applications, the applicator tool 10 can likewise be used for purposes other than cosmetic or medical applications. For example, in implementations the applicator tool 10 can be used in painting applications, such as to paint houses, other buildings, cars, or other objects. The applicator tool 10 can still further be used in artistic applications, such as painting, sculpting, or others.

Structures or features described herein as singular or integrated can in implementations be plural or distributed, and structures or features described as multiple or distributed can in implementations be combined. The scope of the present teachings is accordingly intended to be limited only by the following claims.

What is claimed is:

1. An applicator tool, comprising:
a support configured to receive and enclose a user's hand; and
a set of applicator pads, attached to the base, the set of applicator pads being adapted to
acquire at least one application material, and
apply the at least one application material to a skin area.

2. The tool of claim 1, wherein the support comprises a glove.

3. The tool of claim 2, wherein the glove comprises at least one finger section.

4. The tool of claim 3, wherein the at least one finger section comprises a plurality of finger sections.

5. The tool of claim 1, wherein the set of applicator pads comprise a sponge material.

6. The tool of claim 1, wherein the set of applicator pads are attached to the base using at least one of
adhesive,
stitching, or
opposed hook and loop contact surfaces.

7. The tool of claim 1, wherein the at least one application material comprises a topically applied medical material.

8. The tool of claim 1, wherein the topically applied medicine comprises at least one of
an analgesic material,
an antibiotic material, or
a self-curing bandage material.

9. The tool of claim 1, wherein the at least one application material comprises at least one cosmetic material.

10. The tool of claim 9, wherein the at least one cosmetic material comprises at least foundation material.

11. The tool of claim 1, wherein at least two of the set of applicator pads comprise an applicator pad having different shapes.

12. The tool of claim 1, wherein the base is made of at least one of
latex material,
rubber material, or
vinyl material.

13. The tool of claim 1, wherein the support comprises an interior liner material.

14. A method of applying material, comprising:
acquiring the material in at least one applicator pad of a support enclosing a user's hand; and
applying the material to a selected area of the user's skin by contacting the at least one applicator pad to the selected area.

15. The method of claim 14, wherein the support comprises a glove.

16. The method of claim 14, wherein the material comprises a medical material.

17. The method of claim 14, wherein the material comprises a cosmetic material.

18. The method of claim 14, wherein the selected area comprises a selected area of the face.

19. The method of claim 14, further comprising moistening the at least one applicator pad before the acquiring.

20. The method of claim 14, further comprising washing the at least one applicator pad in a machine after the applying.
